(19) United States
(12) Patent Application Publication
(10) Pub. No.: US 2008/0160082 A1
(43) Pub. Date: Jul. 3, 2008

(54) Controlled release compositions comprising Nimesulide

(75) Inventors: Amarjit Singh, New Delhi (IN); Rajesh Jain, New Delhi (IN)

Correspondence Address: LADAS & PARRY LLP, 26 WEST 61ST STREET, NEW YORK, NY, 10023, US

(73) Assignee: PANACEA BIOTEC LIMITED

(21) Appl. No.: 11/978,162

(22) Filed: Oct. 26, 2007

Related U.S. Application Data
(63) Continuation of application No. 10/089,020, filed on Mar. 27, 2003.

(30) Foreign Application Priority Data
Sep. 28, 1999 (IN) 1297/DEL/99

(51) Int. Cl. A61K 9/22; A61K 31/18

(52) U.S. Cl. 424/468; 514/605

(57) ABSTRACT

A controlled release pharmaceutical composition for peroral administration including a single unit fast release fraction and a single unit extended release fraction which includes nimesulide as an active drug upto 99% w/w of the composition, one or more release controlling materials from 0.1% to 99% w/w of the composition and pharmaceutical excipients from 0% to 90% w/w of the composition. The nimesulide is present in the fast release fraction and in the extended release fraction.

### PARENT CASE TEXT

This application is a continuation application of U.S. Ser. No. 10/089,020 filed on Mar. 25, 2002 which is a National Phase application of PCT International Application No. PCT/IN00/00094 filed on Sep. 27, 2000 claiming a priority from Indian Patent Application No. 1297/DEL/99 dated Sep. 28, 1999; the contents of which are hereby incorporated by reference into the present application.

### FIELD OF THE INVENTION

The present invention relates to a controlled release composition of Nimesulide. The composition is related to a once-a-day dosage forms which are very useful in treatment of chronic diseases such as arthritis.

### TECHNICAL BACKGROUND OF THE INVENTION

Nimesulide is a nonsteroidal anti-inflammatory drug (NSAID) that also has antipyretic and analgesic properties. The compound is weakly acidic (pKa=6.5) and differs from other NSAIDs in that its chemical structure contains a sulfonanilide moiety as the acidic group (FIG. 1) (Magni E, Nimesulide an overview, Drug Invest 1991; 3 Suppl. 2: 1-3).

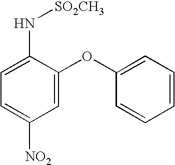

### FIG. 1

The therapeutic effects of NSAIDs are largely the result of their ability to inhibit prostaglandin synthesis via inhibition of cyclooxygenase. Unfortunately, this effect is also responsible for the inhibition of gastroprotective prostaglandins, which leads to gastrointestinal intolerance.

In vitro, Nimesulide is a relatively weak inhibitor of prostaglandin synthesis and appears to exert its effects through a variety of mechanisms. (Magni E., The effect of nimesulide on prostanoid formation, Drugs 1993, 46 Suppl. 1:10-4) Indeed, the mechanism of action of this drug is more complex than previously thought and may involve interference with the production/action of mediators other than prostaglandins such as enzymes, toxic oxygen derivatives, cytokines, platelet-activating factor (PAF) and histamine.

The anti-inflammatory, analgesic and antipyretic activities of Nimesulide, a non-steroidal anti-inflammatory drug (NSAID) of the sulfonanilide class, have been demonstrated in a number of experimental models and in numerous clinical trials. Nimesulide has exhibited potency similar to or greater than that of indomethacin, diclofenac, piroxicam and ibuprofen in standard animal models of inflammation such as carrageenan-induced rat paw oedema and inflammation, ultraviolet light-induced erythema in guinea-pigs and adjuvant arthritis in rats. The analgesic potency in nimesulide was similar to that of ibuprofen and less than that of indomethacin in an acetic acid writhing test in rats, and acetic acid and acetylcholine writhing tests in mice. Nimesulide has shown superior antipyretic potency to indomethacin, ibuprofen, aspirin and paracetamol (acetaminophen) in rats with yeast-induced fever.

Nimesulide is a relatively weak inhibitor of prostaglandin synthesis in vitro and appears to exert its effects through a variety of mechanisms including free-radical scavenging, effects on histamine release, the neutrophil myeloperoxidase pathway, bradykinin activity, tumour necrosis factor-α release, cartilage degradation, metalloprotease synthesis, phosphodiesterase type IV inhibition, platelet aggregation and synthesis of platelet activating factor. Animal studies have suggested that nimesulide is less ulcerogenic than aspirin, indomethacin, naproxen, piroxicam and ibuprofen. Nimesulide appears to have little effect on renal prostaglandin synthesis in rats.

After oral administration of nimesulide 50 to 200 mg to healthy adult volunteers, peak serum concentrations of 1.98 to 9.85 mg/L are achieved within 1.22 to 3.17 hours. Compared with values obtained with oral drug administration, peak serum concentrations are slightly lower (2.14 to 2.32 mg/L) and are achieved more slowly (3 to 4.58 h) after rectal administration of nimesulide 100 and 200 mg. Oral drug absorption is nearly complete and concomitant administration of food may decrease the rate, but not the extent of absorption of nimesulide. The drug is extensively bound (99%) to plasma proteins and has an estimated apparent volume of distribution of 0.19 to 0.35 L/kg following oral administration.

In children, nimesulide suspension, granules and suppositories are more effective than placebo and are at least as effective as paracetamol, diclofenac, naproxen, lysine acetylsalicylate, mefenamic acid, ketoprofen and dipyrone in reducing the pain, inflammation and fever associated with respiratory tract infection, postoperative pain and musculoskeletal injury.

Nimesulide has been well tolerated by both young and elderly adults and children in 2 large postmarketing surveillance surveys. As with other NSAIDs, the most common adverse effects are gastrointestinal disturbances (epigastralgia, heartburn, nausea, diarrhea and vomiting in 5.1 to 8.5% of patients), dermatological reactions (rash, pruritus; 0.2 to 0.6%) and central nervous system effects (dizziness, somnolence, headache; 0.3 to 0.4%). Withdrawal rates associated with short term (up to 30 days) nimesulide treatment range from 1.1 to 2.2% in adult, elderly and pediatric patients.

Available data indicate that the gastrointestinal tolerability of nimesulide in adults and children is similar to that of other NSAIDs. The rate of endoscopically verified gastroduodenal irritation with nimesulide appears to be similar to that with placebo and diclofenac and less than that with indomethacin. The drug is well tolerated by most patients intolerant of aspirin and/or other NSAIDs and by patients with asthma.

The literature surveys shows that different dosage forms reported for nimesulide are tablets, granules, suppositories and suspensions (Drugs 48 (3): 431-454, 1994) and lately our group has patented transdermal (U.S. Pat. No. 5,688,829) and intramuscular injection (U.S. Pat. No. 5,716,609) formulations. The reported dosage forms have to be administered twice-a-day based on biological half life of nimesulide. The usual oral/rectal dosage of nimesulide in adults is 100 to 200 mg twice daily, orally. For treatment of chronic diseases like arthritis the twice daily dosing regimen is difficult to comply with.

One approach to improve the possible non-compliance with the regimen is to develop controlled release dosage form for nimesulide. The once-a-day dosage form is expected to significantly increase the dosing convenience and patient compliance. However, controlled release once-a-day dosage form of nimesulide has not been reported so far.

Controlled release compositions for oral use in the form of matrix type monolithic tablets, beads, capsules and coated tablets are known. However poorly soluble drugs like nimesulide are known to give erratic and variable release under in-vivo conditions from such dosage forms.

One approach to formulate modified release dosage forms of NSAIDs is described in PCT Pub. No. WO9912524, wherein a unit dosage form comprising two fractions (i) a first quick release fraction, and (ii) a second fraction containing coated delayed release multiple units is described. However, such dosage forms having different fractions and coated multiple units are difficult to prepare and very cost intensive. Moreover compression of such coated multiple units into tablets cause fracturing of the coat layer, thereby causing loss of reproducibility.

U.S. Pat. No. 5,788,987 (Busetti et al.) describes a time-specific controlled release dosage form. Such dosage forms are designed to provide delayed release of the active ingredient rather than extended release. Such formulations are not suitable for day long management of the disease.

### SUMMARY OF THE INVENTION

By expenditure of considerable intellectual effort and careful experimentation the inventors have discovered that nimesulide can be formulated into a controlled release once-a-day oral dosage form.

Such dosage forms provide extended release of nimesulide in-vivo when given once daily with reproducible bioavailability. Further the release of such dosage forms is not affected by pH changes in the gastrointestinal system.

The composition in accordance with present invention comprises a controlled release pharmaceutical composition of Nimesulide which comprises nimesulide as an active drug from 5% to 95% w/w of the composition in micronized form, one or more release sustaining materials from 2% to 95% w/w of the composition and pharmaceutical excipients from 0% to 90% w/w of the composition.

Preferably the composition in accordance with the present invention comprises nimesulide as an active drug from 20% to 70% w/w of the composition, one or more sustaining materials from 5% to 65% w/w of the composition and pharmaceutical excipients from 10% to 70% w/w of the composition.

More preferably the composition in accordance with the present invention comprises nimesulide as an active drug from 40% to 60% w/w of the composition, one or more sustaining materials from 8% to 20% w/w of the composition and pharmaceutical excipients from 30% to 60% w/w of the composition.

### DETAILED DESCRIPTION OF INVENTION

In accordance with the present invention there is disclosed a controlled release composition of Nimesulide.

The composition in accordance with present invention comprises a controlled release pharmaceutical composition of Nimesulide which comprises nimesulide as an active drug from 5% to 95% w/w of the composition, one or more sustaining materials from 2% to 95% w/w of the composition and pharmaceutical excipients from 0% to 90% w/w of the composition. In another aspect, such compositions contain nimesulide in micronized form having average particle size below 5 microns.

Preferably the composition in accordance with the present invention comprises nimesulide as an active drug from 20% to 70% w/w of the composition, one or more sustaining materials from 5% to 65% w/w of the composition and pharmaceutical excipients from 10% to 70% w/w of the composition.

More preferably the composition in accordance with the present invention comprises nimesulide as an active drug from 40% to 60% w/w of the composition, one or more sustaining materials from 8% to 20% w/w of the composition and pharmaceutical excipients from 30% to 60% w/w of the composition.

In a preferred embodiment of the invention the composition consists of bilayer tablets wherein the active agent may be present in one or both layers. The bilayer tablets may be coated or uncoated. The coating may be semi-permeable type membrane. Further, the semi-permeable coat may have an orifice drilled through it on the drug layer side to provide passage for constant release of drug.

In another aspect of the invention the coating may be of microporous type through which the drug release takes place at constant rate.

In another aspect of the invention the bilayer tablet dosage form may have a first layer which gives fast release of the drug, and a second layer which gives extended release of the drug.

The first fast release layer comprises materials like disintegrants, fillers, rapidly soluble/dispersible excipients and wetting agents. The second extended release layer comprises sustaining polymers, binders, wetting agents and fillers.

The sustaining polymers preferably are hydrophilic in nature and present in a blend of fast and slow hydrating polymers. The sustaining materials are selected from the group comprising cellulose and cellulose derivatives, waxes, carbomers, polyalkylene polyols, polycarbophils, methacrylic acid derivatives, gelatins, gums, polyethylene oxides.

The sustaining materials comprise materials which are non-toxic and pharmaceutically acceptable. These may be natural, semi-synthetic, synthetic or man-modified. Suitable materials include cellulose and cellulose derivatives like microcrystalline cellulose, methyl cellulose, ethyl cellulose, hydroxypropyl methylcellulose, hydroxypropyl cellulose, cellulose acetate phthalate, cellulose acetate, cellulose acetate butyrate, cellulose acetate propionate, cellulose acetate trimellitate, cellulose carboxymethyl ethers and their salts, hydroxypropylmethyl cellulose phthalate, hydroxypropylmethyl cellulose acetate succinate; polyethylene; polyquaternium-1; polyvinyl acetate (homopolymer); polyvinyl acetate phthalate; propylene glycol alginate; polyvinylmethacrylate/maleic anhydride (PVM/MA) copolymer; polyvinylpyrrolidone (PVP)/dimethiconylacrylate/polycarbamyl/polyglycolester; PVP/dimethylaminoethyl methacrylate copolymer; PVP/dimethylaminoethyl methacrylate/polycarbamyl polyglycol ester; PVP/polycarbamyl polyglycol ester; polyvinylpyrrolidone/vinyl acetate (PVP/VA) copolymer; lanolin and lanolin derivatives; glyceryl monostearate; stearic acid; paraffins; beeswax; carnauba wax; tribehenin; polyalkylene polyols like polyethylene glycols; gelatin and gelatin derivatives; alginates; carbomers; polycarbophils; methacrylic acid copolymers; carrageenans; pectins; chitosans; cyclodextrins; lecithins; natural and synthetic gums containing galactomannans like xanthan gum, tragacanth, acacia, agar, guar gum, and the like.

Pharmaceutical excipients as used in the composition are selected from the group of excipients generally used by persons skilled in the art e.g. fillers, bulking agent, colorants, stabilizers, preservatives, lubricants, glidants, chelating agents and the like.

Preferably the composition also comprises release modifiers. Such release modifiers are selected from the group comprising wetting agents, solubilizers, surfactants, plasticizers, solvents, pore formers, pH modifiers and tonicity adjusting agents.

Suitable example of such ingredients include reaction products of natural and hydrogenated vegetable oils and ethylene glycol e.g. polyoxyethylene glycolated natural or hydrogenated castor oil such as those available under the trade name CREMOPHOR®. Other suitable products include polyoxyethylene sorbitan fatty acid esters e.g. of the type available under the trade name TWEEN®; polyoxyethylene fatty acid esters e.g. MYRJ® and CETIOL® HE; polyoxyethylene polyoxypropylene copolymers e.g. PLURONIC® and polyoxyethylene polyoxypropylene block copolymers e.g. POLOXAMER®; dioctylsodiumsulfosuccinate; sodium lauryl sulphate; propylene glycol mono- and di-fatty acid esters e.g. MIGLYOL® 840; bile salts e.g. alkali metal salts e.g. sodium taurocholate; polyethylene glycols; propylene glycol; triacetin; diacetin; diethyl phthalate; dibutyl phthalate; castor oil; triethyl citrate; dibutyl sebacate; sodium chloride; potassium chloride; lactose; mannitol; sucrose; sorbitol; sodium hydroxide; potassium hydroxide; sodium bicarbonate; sodium citrate; citric acid; hydrochloric acid; lactic acid; tartaric acid; malic acid and the like.

The calculation of dose of nimesulide for once-a-day controlled release dosage form was done on the basis of its pharmacokinetic parameters using the following equation:

Dose=C_{P}×V_{d}×Kₑₗ×T

C_{P}=Effective plasma concentration, 3.0 mg/L

V_{d}=Apparent Volume of distribution, 15.6 L

Kₑₗ=Elimination Rate constant, 0.166 h⁻¹

T=Desired Duration of action, 24 hrs

Based on the above equation the dose was calculated to be 207.0 mg.

The compositions of the present invention have another added advantage that once-a-day dosage form of Nimesulide may be combined with another suitable long-acting drug to have synergistic activity. The other drug may be present in non-controlled release form. Such drugs may be selected from following categories:

(i) Antihistaminics e.g. Cetirizine Dihydrochloride.

(ii) Antispasmodics e.g. Pitofenone Hydrochloride, Hyoscine Hydrobromide.

(iii) Antiasthmatics e.g. Ketotifen, Salbutamol.

The foregoing examples are illustrative embodiments of the invention and are merely exemplary. A person skilled in the art may make variations and modifications without depending from the spirit and scope of the invention. All such modifications and variation are intended to be included within the scope of the invention as discuss in this specification.

### EXAMPLE-1

### Controlled Release (CR) Matrix Tablet Type

| S. No. | Ingredient | mg/tablet |
|---|---|---|
| 1. | Nimesulide (micronized) | 200.0 |
| 2. | Lactose | 73.0 |
| 3. | Hydroxypropyl methylcellulose | 70.0 |
| 4. | Magnesium stearate | 3.5 |
| 5. | Purified talc | 3.5 |

### Procedure:

Blend (1), (2), (3), (4) and (5) after sifting through mesh no. 30 (BSS). Compress into tablets.

The dissolution release profile of Nimesulide CR tablets based on example 1 is given below in Table-1:

TABLE 1

| Time | | Mean | SD |
|---|---|---|---|
| 30 | mins. | 4.2 | ±1.36 |
| 1 | hr | 7.9 | ±1.02 |
| 2 | hrs | 16.4 | ±1.74 |
| 3 | hrs | 25.8 | ±1.28 |
| 4 | hrs | 34.2 | ±1.71 |
| 6 | hrs | 50.8 | ±2.44 |
| 8 | hrs | 65.9 | ±1.86 |
| 10 | hrs | 74.9 | ±0.97 |
| 12 | hrs | 85.8 | ±2.34 |
| 14 | hrs | 93.5 | ±2.49 |
| 16 | hrs | 96.7 | ±2.16 |
| 18 | hrs | 97.1 | ±1.08 |
| 19 | hrs | 98.8 | ±1.32 |

The dissolution profile as given in Table 1 of Nimesulide CR tablet should not be construed to limit the scope of the invention. Variations to the dissolution profile can be possible depending upon the dosage requirements without departing from the spirit of the invention.

### EXAMPLE-2

### Extended Release Membrane Diffusion Controlled Tablet Type

| S. No. | Ingredient | mg/tablet |
|---|---|---|
| 1. | Nimesulide (micronized) | 200.0 |
| 2. | Microcrystalline cellulose | 60.0 |
| 3. | Lactose | 60.0 |
| 4. | Maize starch | 10.0 |
| 5. | Purified talc | 3.5 |
| 6. | Ethyl cellulose (as aqueous dispersion) | 10.0 |
| 7. | Polyethylene glycol | 3.5 |

### Procedure:

Blend (1), (2), and (3) and granulate with maize starch paste and dry the granules. Sift through mesh no. 22 (BSS). Lubricate with Talc. Compress into tablets. Coat the tablets with Ethyl cellulose using Polyethylene glycol as a channel former.

### EXAMPLE-3

### Sustained Release Bead Type

| S. No. | Ingredient | quantity (mg) |
|---|---|---|
| 1. | Non - Pareil Beads | 347.0 |
| 2. | Nimesulide | 200.0 |
| 3. | Mannitol | 30.0 |
| 4. | Lactose | 30.0 |
| 5. | Polyvinyl pyrrolidone | 20.0 |
| 6. | Purified talc | 15.0 |
| 7. | Ethyl cellulose | 7.0 |
| 8. | Diethyl phthalate | 1.4 |

### Procedure:

Coat the Non-pareil beads with blend of (2), (3) and (4) using (5) as a binder in a conventional or fluidized bed coater. Talc may be dusted onto the beads. Final coating is given with Ethyl cellulose using (8) as plasticizer.

### EXAMPLE-4

### Osmotically Controlled Constant Release Type Device

| | | |
|---|---|---|
| S. No. | Ingredient | mg/tablet |

Upper Layer

| | | |
|---|---|---|
| 1. | Nimesulide (micronized) | 200.0 |
| 2. | Sodium hydroxide | 15.0 |
| 3. | Lactose | 34.0 |
| 4. | Sodium chloride | 30.0 |
| 5. | Polyvinyl pyrrolidone | 6.0 |
| 6. | Polyethylene oxide | 1.5 |

Lower Layer

| | | |
|---|---|---|
| 7. | Polyethylene oxide | 22.0 |
| 8. | Hydroxypropyl methylcellulose | 1.8 |
| 9. | Sodium chloride | 20.0 |
| 10. | Dichloromethane | q.s. (Lost in processing) |

Semi-permeable Coat

| | | |
|---|---|---|
| 11. | Cellulose acetate | 30.0 |
| 12. | Triacetin | 1.0 |
| 13. | Acetone | q.s. (Lost in procesing) |
| 14. | Water | q.s. (Lost in processing) |

### Procedure:

Blend finely powdered (1), (2), (3), (4) and (6). Granulate with aqueous solution of (5). Granulate the blend of (7) and (9) with dispersion of (8) in (10). Compress the two granulates into bilayer tablets and coat with the dispersion of (11) and (12) in aqueous acetone. Finally, drill a hole in the drug layer (Upper layer) through which the drug is released in a controlled fashion due to osmotic pressure.

The dissolution release profile of Nimesulide CR tablets based on example 4 is given below in Table-2:

TABLE 2

| Time | | Mean | SD |
|---|---|---|---|
| 2 | hours | 5.16 | ±0.53 |
| 4 | hours | 16.75 | ±1.68 |
| 6 | hours | 34.90 | ±2.26 |
| 8 | hours | 45.75 | ±2.26 |
| 10 | hours | 56.00 | ±4.36 |
| 12 | hours | 67.85 | ±4.40 |
| 14 | hours | 79.16 | ±5.03 |
| 14 | hours | 90.25 | ±3.68 |
| 18 | hours | 101.16 | ±3.53 |

### EXAMPLE-5

### Coated Capsule Type

| S. No. | Ingredients | mg/capsule |
|---|---|---|
| 1. | Nimesulide (micronized) | 200.0 |
| 2. | Microcrystalline cellulose | 88.4 |
| 3. | Lactose | 70.0 |
| 4. | Polyvinyl pyrrolidone | 7.0 |
| 5. | Magnesium stearate | 3.9 |
| 6. | Ethyl cellulose | 20.0 |
| 7. | Polyethylene glycol | 0.7 |
| 8. | Alcohol:Dichloromethane (1:2) | q.s. (Lost in processing) |
| 9. | Empty gelatin capsule (Size ‘1’) | |

### Procedure:

Blend (1), (2), (3), (4) and (5) and fill into empty gelatin capsule size ‘1’. Coat the capsule with dispersion of (6) and (7) in (8).

### EXAMPLE-6

### pH Dependent Delayed Release Type

| S. No. | Ingredients | mg/tablet |
|---|---|---|
| 1. | Nimesulide (micronized) | 100.0 |
| 2. | Microcrystalline cellulose | 150.0 |
| 3. | Lactose | 76.0 |
| 4. | Polyoxyl 40 hydrogenated castor oil | 7.0 |
| 5. | Polyvinyl pyrrolidone | 10.0 |
| 6. | Magnesium stearate | 3.5 |
| 7. | Purified talc | 3.5 |
| 8. | Cellulose acetate phthalate | 28.0 |
| 9. | Diethyl phthalate | 2.0 |
| 10. | Water | q.s. (Lost in processing) |
| 11. | Alcohol:Dichloromethane (1:2) | q.s. (Lost in processing) |

### Procedure:

Granulate the blend of (1), (2) and (3) with solution of (4) and (5) in water. Blend the granules with (6) and (7). Compress into tablets. Coat with the dispersion of (8) and (9) in (11).

### EXAMPLE-7

### Timed Release Bead Type

| | | quantity | quantity | quantity |
|---|---|---|---|---|
| S. No. | Ingredients | (mg) | (mg) | (mg) |
| 1. | Nimesulide (micronized) | 100.0 | 100.0 | 100.0 |
| 2. | Microcrystalline | 200.0 | 200.0 | 200.0 |
| | cellulose | | | |
| 3. | Lactose | 50.0 | 42.0 | 35.0 |
| 4. | Polyvinyl pyrrolidone | 10.0 | 10.0 | 10.0 |
| 5. | Water | q.s. | q.s. | q.s. |
| 6. | Ammonio methacrylate | 10.0 | 18.0 | 25.0 |
| | copolymer Type B | | | |
| | (Eudragit ® RS) | | | |
| 7. | Diacetin | 0.5 | 0.5 | 0.5 |
| 8. | Water:Acetone (1:9) | q.s. | q.s. | q.s. |

### Procedure:

In this composition, three types of beads are prepared which are coated with different amounts of (6) to give a timed profile of the drug. Beads are prepared by blending and spheronizing (1), (2) and (3) using aqueous solution of (4). The dried beads are coated with dispersion of (6) and (7) in (8). The three different beads are blended together in a fixed ratio to obtain the required release profile.

### EXAMPLE-8

### Nimesulide CR+Cetirizine Bilayered Tablets

| | | |
|---|---|---|
| S. No. | Ingredients | mg/tablet |

Nimesulide Layer

| | | |
|---|---|---|
| 1. | Nimesulide (micronized) | 200.0 |
| 2. | Lactose | 106.5 |
| 3. | Polyoxyl 40 hydrogenated castor oil | 2.0 |
| 4. | Hydroxypropyl methylcellulose | 31.5 |
| 5. | Magnesium stearate | 2.0 |
| 6. | Colloidal silicon dioxide | 2.0 |

Cetirizine Layer

| | | |
|---|---|---|
| 7. | Cetirizine dihydrochloride | 10.0 |
| 8. | Lactose | 105.0 |
| 9. | Microcrystalline cellulose | 25.0 |
| 10. | Starch | 5.0 |
| 11. | Croscarmellose sodium | 3.0 |
| 12. | Magnesium stearate | 2.0 |

### Procedure:

Blend the components of the two layers separately and compress into bilayer tablets.

### EXAMPLE-9

### Osmotically Controlled Constant Release System

| | | |
|---|---|---|
| S. No. | Ingredients | mg/tablet |

Active Layer

| | | |
|---|---|---|
| 1. | Nimesulide (micronized) | 200.0 |
| 2. | Polyethylene oxide | 116.5 |
| 3. | Hydroxypropyl methylcellulose | 10.0 |
| 4. | Sodium chloride | 10.0 |
| 5. | Magnesium stearate | 2.5 |

Push layer

| | | |
|---|---|---|
| 6. | Polyethylene oxide | 140.0 |
| 7. | Sodium chloride | 50.0 |
| 8. | Hydroxypropyl methylcellulose | 9.5 |
| 9. | Magnesium stearate | 0.5 |
| 10. | Iron oxide red | 1.0 |

Functional coating

| | | |
|---|---|---|
| 11. | Cellulose acetate | 45.0 |
| 12. | Polyethylene glycol | 5.0 |
| 13. | Acetone | (Lost in processing) |

Non-functional coating

| | | |
|---|---|---|
| 14. | Titanium dioxide | 2.0 |
| 15. | Hydroxypropyl methylcellulose | 6.0 |
| 16. | Purified Talc | 2.0 |
| 17. | Polyethylene glycol - 400 | 2.0 |
| 18. | Isopropyl alcohol | (Lost in processing) |
| 19. | Dichloromethane | (Lost in processing) |

### Procedure:

Blend (1), (2), (3), (4) and (5) in a double cone blender. Separately blend (6), (7), (8) (9)

and (10). Compress into bilayer tablet using a suitable compression machine. Coat the tablets with the dispersion of (1) and (12) in (13). The tablets are further coated with the dispersion of (14), (15), (16), (17) in mixture of (18) and (19).

### EXAMPLE-10

### Bilayer Tablets Having One Fast Release Layer and One Extended Release Layer

| | | |
|---|---|---|
| S. No. | Ingredients | mg/tablet |

Fast Release layer

| | | |
|---|---|---|
| 1. | Nimesulide (micronized) | 100.0 |
| 2. | Lactose | 151.5 |
| 3. | Starch | 37.6 |
| 4. | Colloidal silicon dioxide | 11.0 |
| 5. | Povidone K-30 | 8.5 |
| 6. | Docusate sodium | 6.8 |
| 7. | Polysorbate 80 | 1.0 |
| 8. | Magnesium stearate | 1.6 |
| 9. | Croscarmellose sodium | 22.0 |
| 10. | Water | (Lost in processing) |

Extended Release Layer

| | | |
|---|---|---|
| 11. | Nimesulide (micronized) | 100.0 |
| 12. | Lactose | 200.0 |
| 13. | Hydroxypropyl methylcellulose K100LV | 23.0 |
| 14. | Hydroxypropyl methylcellulose K4MCR | 100.0 |
| 15. | Povidone K-30 | 9.0 |
| 16. | Docusate sodium | 4.5 |
| 17. | Magnesium stearate | 4.5 |
| 18. | Colloidal silicon dioxide | 4.5 |
| 19. | Sodium lauryl sulphate | 4.5 |
| 20. | Isopropyl alcohol | (Lost in processing) |

### Procedure:

Blend 1: Blend (1), (2), (3) and (4) and granulate with solution of (5), (6) and (7) in (10). Dry the granules and blend with (8) and (9).

Blend 2: Blend (11), (12), (13) and (14) and granulate with solution of (15) and (16) in (20). Dry the granules and mix with (17), (18) and (19). Compress into bilayer tablets using a suitable compression machine.

### EXAMPLE-11

### Bilayer Tablets Having One Fast Release Layer Containing Drug in Complexed Form and One Extended Release Layer

| | | |
|---|---|---|
| S. No. | Ingredients | mg/tablet |

A. Fast Release layer

| | | |
|---|---|---|
| 1. | Nimesulide (micronized) | 100.0 |
| 2. | β-cyclodextrin | 400.0 |
| 3. | Starch | 70.0 |
| 4. | Povidone K-30 | 7.5 |
| 5. | Croscarmellose sodium | 20.0 |
| 6. | Magnesium stearate | 2.5 |

B. Extended Release Layer

| | | |
|---|---|---|
| 7. | Nimesulide (micronized) | 100.0 |
| 8. | Lactose | 200.0 |
| 9. | Hydroxypropyl methylcellulose K100LV | 23.0 |
| 10. | Hydroxypropyl methylcellulose K4MCR | 100.0 |
| 11. | Povidone K-30 | 9.0 |
| 12. | Magnesium stearate | 4.5 |
| 13. | Colloidal silicon dioxide | 4.5 |
| 14. | Docusate sodium | 4.5 |

### Procedure:

### Layer-1

1. Mix (1) and (2), co-mill under specific conditions favouring complexation using ball mill to prepare a complex.
2. Mix complex of step 1 with (3) and granulate with a solution of (4) in water.
3. Dry the granules at 40°-50° C.
4. Size the granules and mix with (5) and (6).

### Layer-II

1. Mix (7), (8), (9) and (10). Granulate with a solution of (11) and (14).
2. Dry the granules at 40°-50° C.
3. Size the granules and mix with (12) and (13).
4. Compress the two layers into bilayered tablets using suitable compression machine.

We claim:

1. A controlled release pharmaceutical composition for peroral administration comprising of a single unit fast release fraction and a single unit extended release fraction which comprises nimesulide as an active drug upto 99% w/w of the composition, one or more release controlling materials from 0.1% to 99% w/w of the composition and pharmaceutical excipients from 0% to 90% w/w of the composition, said nimesulide being present in the fast release fraction and in the extended release fraction.

2. A controlled release pharmaceutical composition of nimesulide as claimed in claim 1, which comprises nimesulide as an active drug from 20% to 70% w/w of the composition, one or more release controlling materials from 5% to 65% w/w of the composition and pharmaceutical excipients from 10% to 70% w/w of the composition.

3. A controlled release pharmaceutical composition of nimesulide as claimed in claim 1, wherein the release controlling materials are selected from a group comprising cellulose and cellulose derivatives, waxes, carbomers, polyalkylene polyols, polycarbophils, methacrylic acid copolymers, gelatins, gums and polyethylene oxides or a combination thereof.

4. The composition as claimed in claim 1, wherein the fast release fraction, the extended release fraction or both further comprise release modifiers selected from a group comprising wetting agents, solubilizers, surfactants, plasticizers, pore formers, pH modifiers and tonicity adjusting agents or a combination thereof.

5. A controlled release pharmaceutical composition as claimed in claim 1, which is a gastroretentive system wherein the residence time of the drug is increased in the stomach, duodenum, jejunum or ileum.

6. The composition as claimed in claim 5, wherein gastroretention of nimesulide is achieved by mucoadhesion, flotation, reducing gastrointestinal motility or a combination thereof.

7. The composition as claimed in claim 6, wherein the extended release fraction comprises polymers having affinity for gastrointestinal mucosa, said polymers selected from a group comprising polycarbophils, carbomers, alginates, cellulose and cellulose derivatives, chitosan, gums and lecithins or a combination thereof to achieve mucoadhesion.

8. The composition as claimed in claim 6, further comprising in the fast release fraction, extended release fraction or both gas-generating materials selected from a group comprising carbonates and bicarbonates alone or in combination with inorganic and organic acids or a combination thereof to achieve floatation.

9. The composition as claimed in claim 6, wherein the material for reducing gastrointestinal motility is selected from a group comprising fats, fatty acids and transesterification products of fats and fatty acids with polyols or combination thereof.

10. The composition as claimed in claim 1, which is in the form of a tablet comprising of a single unit fast release layer and a single unit extended release layer which comprises nimesulide as an active drug upto 99% w/w of the tablet composition, one or more release controlling materials from 0.1% to 99% w/w of the tablet composition and pharmaceutical excipients from 0% to 90% w/w of the tablet composition, said nimesulide being present in the fast release layer and in the extended release layer.

11. The composition as claimed in claim 2, which is in the form of a tablet comprising of a single unit fast release layer and a single unit extended release layer which comprises nimesulide as an active drug from 20% to 70% w/w of the composition, one or more release controlling materials from 5% to 65% w/w of the composition and pharmaceutical excipients from 10% to 70% w/w of the tablet composition, said nimesulide being present in the fast release layer and in the extended release layer.

12. A controlled release pharmaceutical tablet composition for peroral administration consisting essentially of a single unit fast release layer and a single unit extended release layer which comprises nimesulide as an active drug upto 99% w/w of the tablet composition, one or more release controlling materials from 0.1% to 99% w/w of the tablet composition and pharmaceutical excipients from 0% to 90% w/w of the tablet composition, said nimesulide being present in the fast release layer and in the extended release layer.

13. A process for the manufacture of a controlled release composition for peroral administration as claimed in claim 1, comprising a single unit fast release fraction and a single unit extended release fraction which comprises mixing together nimesulide as an active drug up to 99% w/w of the composition, one or more release controlling materials from 0.1% to 99% w/w of the composition and pharmaceutical excipients from 0% 0 to 90% w/w of the composition said nimesulide being present in the fast release fraction and in the extended release fraction.

14. The composition according to claim 1, wherein the fast release fraction comprises nimesulide and one or more pharmaceutical excipients selected from a group comprising diluents, binders, wetting agents, disintegrants and lubricants; and the extended release fraction comprises nimesulide and release controlling material.

15. A controlled release pharmaceutical tablet composition for peroral administration as claimed in claim 10, comprising a single unit fast release layer and a single unit extended release layer which comprises nimesulide as an active drug upto 99% w/w of the tablet composition, one or more release controlling materials from 0.1% to 99% w/w of the tablet composition and pharmaceutical excipients from 0% to 90% w/w of the tablet composition, wherein the fast release layer comprises nimesulide and one or more excipient(s) selected from a group comprising lactose, starch, colloidal silicon dioxide, polyvinylpyrrolidone, polyoxyethylene sorbitan monostearate, docusate sodium, magnesium stearate and croscarmellose sodium, and the extended release layer comprises nimesulide and one or more component(s) selected from a group comprising lactose, polyvinylpyrrolidone, magnesium stearate, docusate sodium, hydroxypropyl methylcellulose, colloidal silicon dioxide and sodium lauryl sulphate.

16. A controlled release pharmaceutical tablet composition for peroral administration as claimed in claim 12, consisting essentially of a single unit fast release layer and a single unit extended release layer which comprises nimesulide as an active drug upto 99% w/w of the tablet composition, one or more release controlling materials from 0.1% to 99% w/w of the tablet composition and pharmaceutical excipients from 0% to 90% w/w of the tablet composition, wherein the fast release layer comprises nimesulide and one or more excipient(s) selected from a group comprising lactose, starch, colloidal silicon dioxide, polyvinylpyrrolidone, polyoxyethylene sorbitan monostearate, docusate sodium, magnesium stearate and croscarmellose sodium, and the extended release layer comprises nimesulide and one or more component(s) selected from a group comprising lactose, polyvinylpyrrolidone, magnesium stearate, docusate sodium, hydroxypropyl methylcellulose, colloidal silicon dioxide and sodium lauryl sulphate.

17. A controlled release pharmaceutical tablet composition as claimed in claim 12, consisting essentially of a single unit fast release layer and a single unit extended release layer which comprises nimesulide as an active drug upto 99% w/w of the tablet composition, one or more release controlling materials from 0.1% to 99% w/w of the tablet composition and pharmaceutical excipients from 0% to 90% w/w of the tablet composition, wherein the fast release layer comprises nimesulide and one or more disintegrant.

18. A controlled release pharmaceutical tablet composition as claimed in claim 17, wherein the disintegrant present in the fast release layer is croscarmellose sodium.

19. A controlled release pharmaceutical tablet composition as claimed in claim 12, consisting essentially of a single unit fast release layer and a single unit extended release layer which comprises nimesulide as an active drug upto 99% w/w of the tablet composition, one or more release controlling materials from 0.1% to 99% w/w of the tablet composition and pharmaceutical excipients from 0% to 90% w/w of the tablet composition, wherein the extended release layer comprises nimesulide and one or more polymer(s).

20. A controlled release pharmaceutical tablet composition as claimed in claim 19, wherein the polymer present in the extended release layer is hydroxypropyl methylcellulose.

21. The composition according to claim 1, further comprising a coating.
